(19) United States
(12) Patent Application Publication
(10) Pub. No.: US 2017/0290852 A1
(43) Pub. Date: Oct. 12, 2017

(54) MICELLE FORMULATIONS OF AMPHOTERICIN B

(71) Applicant: Wisconsin Alumni Research Foundation, Madison, WI (US)

(72) Inventor: Glen S. KWON, Madison, WI (US)

(21) Appl. No.: 15/482,469

(22) Filed: Apr. 7, 2017

Related U.S. Application Data
(60) Provisional application No. 62/320,292, filed on Apr. 8, 2016.
(60) Provisional application No. 62/417,886, filed on Nov. 4, 2016.

(51) Int. Cl. A61K 31/7048; A61K 9/00; A61K 47/28; A61K 31/513; A61K 9/107; A61K 47/24

(52) U.S. Cl. CPC A61K 31/7048; A61K 9/107; A61K 47/24; A61K 47/28; A61K 31/513; A61K 9/0019

(57) ABSTRACT

The present technology relates generally to micelle formulations of amphotericin B that optionally include 5-fluorocytosine and methods of preparing such formulations. Methods of treating fungal infections with the present compositions are also provided.

### CROSS-REFERENCE TO RELATED APPLICATIONS

This application claims priority to U.S. Provisional Patent Application Nos. 62/320,292 and 62/417,886, filed on Apr. 8, 2016 and Nov. 4, 2016, respectively, the entire contents of which are incorporated herein by reference.

### GOVERNMENT RIGHTS

This invention was made with government support under AI10157 awarded by the National Institutes of Health. The government has certain rights in the invention.

### FIELD

The present disclosure relates generally to micelle formulations of amphotericin B (AmB) and methods of treating fungal infections using such formulations. The micelle formulations may include saline without affecting stability of the formulation. The micelle formulations may include other antifungals such as 5-fluorocytosine (5-FC).

### BACKGROUND

The potent antifungal drug amphotericin B (AmB) exerts fungicidal activity by binding to ergosterol, an important cell membrane lipid. Poorly water soluble, AmB tends to self-aggregate, a property that contributes to loss of binding specificity and consequential toxicity. Severe nephrotoxicity has limited its clinical use. Attempts to reduce toxicity have resulted in lipid based formulations that are less nephrotoxic in comparison to the original AmB formulation, Fungizone® (AmB-SD). However, several lipid-based AmB formulations that exhibit decreased renal toxicity possess other disadvantages. Lipid AmB formulations require higher dosing to achieve antifungal efficacy similar to that of AmB-SD. Additionally, they are considerably more costly. Depending on the fungal infection, it may be necessary to treat a patient with AmB for several weeks. Taking this into consideration, the decreased antifungal efficacy compounded with the cost of lipid AmB can act as a barrier to patient accessibility. Furthermore, dose-limiting renal toxicity is still an issue with these improved lipid formulations.

Another potent antifungal drug is the prodrug 5-fluorocytosine (5-FC). The prodrug 5-FC appears to be converted to its biologically active metabolites by cytosine deaminase, an enzyme present in susceptible fungi cells but not in human cells. The metabolites, 5-fluorouridinetriphosphate and 5-fluorodeoxyuridinemonophosphate, exert fungicidal activity by disrupting fungal RNA biosynthesis by preventing the building of certain essential proteins and by inhibiting fungal DNA synthesis. In the United States, 5-FC is dosed orally as a capsule (Ancobon®), however, an infusion solution for IV administration is approved for use in Europe. Side effects of orally administered 5-FC, including myelosuppression and liver damage, may stem from conversion of the prodrug to 5-fluorouracil, a precursor of DNA and protein synthesis inhibitors, by gut microflora. Intravenous (IV) administration of 5-FC may result in less toxicity than oral delivery because it has the potential for less gut exposure and, therefore, reduced nonspecific conversion to 5-fluorouracil. Additionally, IV administration of 5-FC may help prevent variation in bioavailability that occurs across different patient populations. However, any desire to combine the currently available IV formulations of AmB and 5-FC is discouraged by both the AmB-SD and 5-FC preparation instructions that clearly state each infusion solution should not be mixed with other drugs. As illustrated in FIG. 9, such a combination can be deadly, because AmB-SD precipitates in the presence of 5-FC in saline resulting in micron-sized particles that could cause embolism.

### SUMMARY OF THE INVENTION

The present technology provides micellar compositions of AmB for use in treating fungal infections. These micellar compositions exhibit significantly reduced aggregation of AmB compared to simple aqueous suspensions of AmB or AmB-SD. Aggregated AmB, exhibited by existing marketed formulations of AmB, is associated with toxicity to mammalian cells. In contrast, the monomeric form of AmB appears to selectively disrupt fungal cell membranes. The present compositions include a pegylated-phospholipid, amphotericin B, a water-soluble salt of deoxycholate, and optionally saline. Typically, the mole ratio of amphotericin B to pegylated-phospholipid in the present composition is about 1:25 to about 1:200, and the concentration of pegylated-phospholipid is above its critical micelle concentration (CMC). Unlike many AmB formulations with reduced nephrotoxicity, the present compositions are simple to prepare and exhibit similar efficacy to the original AmB-SD formulation.

Surprisingly, even though the present compositions may include saline, AmB is far more deaggregated than in saline alone, which increases aggregation and can lead to precipitation of AmB from solution. In fact, saline, which can reduce the cytotoxicity of AmB to mammalian cells, cannot be concurrently administered with AmB-SD itself due to the potential for precipitation and embolism.

Similarly, the combined delivery of AmB and 5-FC intravenously has been previously precluded due to the potential for precipitation and embolism. However, the present technology provides a composition that includes both AmB and 5-FC that does not have these same drawbacks. These combination delivery compositions may achieve additive to synergistic antifungal efficacy. Although not wishing to be bound by theory, it is believed that the additive to synergistic effects may be attributed to enhanced uptake of 5-FC resulting from AmB-induced membrane disruption and sequential drug action. In some embodiments, compositions that include both AmB and 5-FC may provide reduced nephrotoxicity of AmB by administering lower doses of the drug and/or administering the drug for a shorter length of time. In some embodiments, the combination of AmB and 5-FC may reduce the likelihood of developing resistance to 5-FC. In some embodiments, the simultaneous IV delivery of AmB and 5-FC may provide antifungal efficacy, ease of preparation, and commercial availability of formulation components. Delivering 5-FC via the IV route may also avoid unpredictable bioavailability (e.g., in patients with HIV/AIDS who have achlorhydria), and, at certain doses in patients without renal insufficiency, could remove the need for serum concentration monitoring. IV delivery also presents an alternative option to treat neonates, patients who are unable to swallow medication, and patients who are allergic to excipients in Ancobon®, i.e. patients to whom oral delivery may not be feasible.

The present technology also provides methods of treatment in which an effective amount of any of the present compositions may be administered to a mammal suffering from a fungal infection, in particular, a human suffering from a fungal infection. In some embodiments, the composition may be administered by an IV route.

### BRIEF DESCRIPTION OF THE DRAWINGS

FIG. 1. Reformulation procedure for AmB-SD with PEG-DSPE micelles in saline.

FIG. 2. UV-visible absorption spectra of AmB-SD alone and with PEG-DSPE micelles in 0.9% NaCl-USP. Samples were concentrated to 0.1 mg/mL AmB and diluted 10-fold in 0.9% NaCl-USP for analysis.

FIGS. 3A and 3B. Z-average particle diameter of AmB-SD alone and with PEG-DSPE micelles in 0.9% NaCl-USP immediately after sample preparation (FIG. 3A) and four hours after sample preparation (FIG. 3B). Samples were concentrated to 0.1 mg/mL AmB and diluted 5-fold in 0.9% NaCl-USP for analysis.

FIG. 4. Hemolytic activity of AmB-SD alone and with PEG-DSPE micelles. Enlargement of hemolytic activity of AmB-SD with PEG-DSPE micelles (Inset). Data presented as mean±standard deviation (n=3).

FIGS. 5A, 5B and 5C. Renal Toxicity. Study methods schematic (FIG. 5A). BUN increase after treatment (FIG. 5B). Creatinine increase after treatment (FIG. 5C). Data presented as mean±standard error of the mean (n=3).

FIGS. 6A and 6B. ATCC 9763 time kill curves of AmB-SD alone and with PEG-DSPE in 0.9% NaCl-USP at 1 μg/mL and 2 μg/mL AmB (FIG. 6A). K1 time kill curves of AmB-SD alone and with PEG-DSPE in 0.9% NaCl-USP at 2 μg/mL and 4 μg/mL AmB (FIG. 6B). Data presented as mean±standard deviation (n=3).

FIG. 7. Reformulation procedure for AmB-SD with PEG-DSPE micelles in saline plus 5-FC.

FIG. 8. UV-visible absorption spectra of AmB-SD+FC alone and with PEG-DSPE micelles in 0.9% NaCl-USP. Samples were concentrated to 0.1 mg/mL AmB and 9 mg/mL 5-FC and diluted 10-fold in 0.9% NaCl-USP for analysis.

FIG. 9. Z-average particle diameter of AmB-SD+FC alone and with PEG-DSPE micelles in 0.9% NaCl-USP immediately after sample preparation. Samples were diluted 5-fold in 0.9% NaCl-USP for analysis.

FIG. 10. Hemolytic activity of AmB-SD+100 μg 5-FC, AmB-SD with PEG-DSPE micelles+50 μg 5-FC, and AmB-SD with PEG-DSPE micelles+100 μg 5-FC. Data presented as mean±standard deviation (n=3).

FIG. 11. Contrasted hemolytic activity after 30 min of exposure to up to 10 μg/mL AmB+5-FC alone and with PEG-DSPE micelles in 0.9% NaCl-USP. Concentrations of 5-FC are expressed in μg/mL. Data presented as mean±standard deviation (n=3) (p<0.0001).

FIGS. 12A, 12B, and 12C. Gu5 time kill curves of 5-FC, AmB-SD with and without PEG-DSPE micelles, and 5-FC+AmB-SD with and without PEG-DSPE micelles at 0.125 μg/mL AmB and 32 μg/mL 5-FC (FIG. 12A). K1 time kill curves of 5-FC, AmB-SD with and without PEG-DSPE micelles, and 5-FC+AmB-SD with and without PEG-DSPE micelles at 0.125 μg/mL AmB and 16 μg/mL 5-FC (FIG. 12B). SC5314 time kill curves of 5-FC, AmB-SD with and without PEG-DSPE micelles, and 5-FC+AmB-SD with and without PEG-DSPE micelles at 6.25*10⁻² μg/mL AmB and 8 μg/mL 5-FC (FIG. 12C). Data presented as mean±standard deviation (n=2).

FIG. 13. Change in log₁₀ CFU/kidneys of K1 in a neutropenic murine model of disseminated candidiasis expressed in response to no drug (control) and five treatment groups: 5-FC (72 mg/kg), AmB-SD (2 mg/kg), mAmB-90 (2 mg/kg), 5-FC+AmB-SD (72 mg/kg+2 mg/kg), and 5-FC+mAmB-90 (72 mg/kg+2 mg/kg). Control is at zero hour (two hours post-infection). Results presented as mean±standard deviation (n=3).

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The present technology provides pharmaceutical compositions and medicaments comprising any of one of the embodiments of the micelles disclosed herein and a pharmaceutically acceptable carrier or one or more excipients. The compositions may be used in the methods and treatments described herein. The pharmaceutical composition may include an effective amount of any of one of the embodiments of the compositions disclosed herein. In any of the above embodiments, the effective amount may be determined in relation to a subject. “Effective amount” refers to the amount of compound, micelle, or composition required to produce a desired effect. One example of an effective amount includes amounts or dosages that yield acceptable toxicity and bioavailability levels for therapeutic (pharmaceutical) use including, but not limited to, the treatment of fungal infections such as systemic fungal infections. As used herein, a “subject” or “patient” is a mammal, such as a cat, dog, rodent or primate. Typically the subject is a human, and, preferably, a human at risk for or suffering from a fungal infection. The term “subject” and “patient” can be used interchangeably.

In one aspect, the present technology provides micellar compositions of amphotericin B, optionally with saline. The compositions provide reduced toxicity with similar efficacy as compared to AmB-SD. The compositions include pegylated-phospholipid, amphotericin B, and a water-soluble salt of deoxycholate. The concentration of pegylated-phospholipid in the compositions is above its CMC. The CMC is the concentration at which the amphiphilic unimers—here, pegylated phospholipid—aggregate into structured micelles. CMC varies depending on the structure of the unimers.

“Pegylated phospholipid” as used herein refers to a phospholipid that has been conjugated to polyethylene glycol (PEG) block through a small organic moiety (e.g., ethanolamine, serine, glycerol and the like) attached to the phosphate group. The phospholipid portion of the pegylated-phospholipid typically includes a bis C₁₂-C₂₀ fatty acid glyceryl ester, preferably bis C₁₄-C₁₈ fatty acid glyceryl esters. For example, the phospholipid may be phosphatidylethanolamine, phosphatidylglycerol, or phosphatidylserine conjugated to glyceryl esters of stearic acid, palmitic acid or myristic acid. In some embodiments of the present compositions, the pegylated-phospholipid is PEG-di stearoylphosphatidylethanolamine (PEG-DSPE). Various forms of pegylated-phospholipid, including PEG-DSPE, are available commercially, such as from Avanti Polar Lipids (Alabaster, Ala.), or they can be prepared according to methods well known to those of skill in the art. For compositions intended for human treatment, preferably the pegylated phospholipid is produced under current Good Manufacturing Processes (cGMP). The molecular weight of the poly(ethylene glycol) block can be about 1,000 to about 10,000 g/mol, roughly equivalent to about 23 to about 227 ethylene oxide groups. (All polymer molecular weights referred to herein will be understood to be weight average molecular weights.) For example, the molecular weight of the PEG block may be about 1,000 Da, about 1,500 Da, about 2,000 Da, about 2,500 Da, about 3,000 Da, about 3,500 Da, about 4,000 Da, about 4,500 Da, about 5,000 Da, about 5,500 Da, about 6,000 Da, about 6,500 Da, about 7,000 Da, about 7,500 Da, about 8,000 Da, about 9,000 Da, or about 10,000 Da, or a range between and including any two of the foregoing values. For example, in some embodiments, the PEG may be about 3,000 Da to about 7,000 Da. In one embodiment, the PEG is about 5,000 Da. The PEG block can terminate in an alkyl group, such as a methyl group (e.g., a methoxy ether) or any suitable protecting, capping, or blocking group.

AmB (including e.g., AmB-SD) may be present at a variety of concentrations in the present compositions, so long as the concentration allows an effective amount of AmB to be administered to a patient in need thereof. In some embodiments, the present compositions include about 0.5 μg/mL to about 1 mg/mL AmB. For example, the present compositions may include about 1.0 μg/mL, about 2.0 μg/mL, about 3.0 μg/mL, about 4.0 μg/mL, about 5.0 μg/mL, about 6.0 μg/mL, about 7.0 μg/mL, about 8.0 μg/mL, about 9.0 μg/mL, about 0.01 mg/mL, about 0.02 mg/mL, about 0.05 mg/mL, about 0.08 mg/mL, about 0.1 mg/mL, about 0.15 mg/mL, about 0.15 mg/mL, about 0.18 mg/mL, about 0.2 mg/mL, about 0.3 mg/mL, about 0.4 mg/mL, about 0.6 mg/mL, about 0.8 mg/mL, about 1.0 mg/mL or a range between and including any two of the foregoing values. Thus in some embodiments, the present compositions may include about 0.05 mg/mL to about 0.2 mg/mL AmB. In one embodiment, the AmB may be about 0.1 mg/mL. In some embodiments, the AmB may be about 1.0 μg/mL to about 10 μg/mL (including about 6 μg/mL).

The present compositions include water soluble deoxycholate salts, e.g., deoxycholate sodium or potassium. “Water soluble” refers herein to a solubility of at least 100 mg/mL in water at 25° C. and pH 7. In some embodiments the water soluble deoxycholate salt is deoxycholate sodium. The water soluble deoxycholate salt is typically employed at a concentration sufficient to form a suspension of the AmB in aqueous solution. This is particularly important when preparing a stock solution of AmB at, e.g., 5 mg/mL, prior to further dilution for administration. Such stock solutions tend to contain predominantly aggregated forms of AmB. Thus, in some embodiments, the present compositions include about 0.001 mg/mL to about 1 mg/mL water soluble deoxycholate salt. For example, the present compositions may include about 1.0 μg/mL, about 2.0 μg/mL, about 3.0 μg/mL, about 4.0 μg/mL, about 5.0 μg/mL, about 6.0 μg/mL, about 7.0 μg/mL, about 8.0 μg/mL, about 9.0 μg/mL, about 0.01 mg/mL, about 0.02 mg/mL, about 0.05 mg/mL, about 0.08 mg/mL, about 0.1 mg/mL, about 0.15 mg/mL, about 0.15 mg/mL, about 0.18 mg/mL, about 0.2 mg/mL, about 0.3 mg/mL, about 0.4 mg/mL, about 0.6 mg/mL, about 0.8 mg/mL, about 1.0 mg/mL water soluble deoxycholate salt or a range between and including any two of the foregoing values. Thus in some embodiments, the present compositions may include about 0.05 mg/mL to about 0.2 mg/mL water soluble deoxycholate salt. In some embodiments, the present compositions may include about 0.5 μg/mL to about 15 μg/mL water soluble deoxycholate salt. The weight ratio of water soluble deoxycholate salt to AmB may vary, e.g., from about 1:2 to 2:1. Typically the ratio ranges from about 2:3 to 3:2 and may be about 4:5 or even about 1:1. AmB is commercially available as a solid in admixture with deoxycholate sodium (AmB-SD) and phosphate buffer salts (X-Gen).

Surprisingly, it has been discovered that the present compositions may include saline, i.e., dilute aqueous sodium chloride, without precipitating the AmB from solution. Current formulations of AmB such as AmB-SD cannot be prepared with saline as the saline may cause the aggregated AmB to precipitate from solution. Such precipitate has the potential to cause embolism in the patient to which the AmB-SD is being administered. In some embodiments of the present compositions including saline, the saline has about 0.5 wt % to about 1.5 wt % sodium chloride. In certain embodiments the saline has about 0.9 wt % sodium chloride.

Surprisingly, it has been discovered that the micellar compositions of the present technology actually act on aqueous suspensions of AmB to deaggregate the AmB. Therefore, even the saline-containing compositions of the present technology contain significantly reduced amounts of aggregated AmB compared to non-saline aqueous suspensions of AmB. The amount of aggregated AmB varies with the mole to mole ratio of AmB to pegylated-phosphlipid. The present compositions typically contain at least a 1:20 mole to mole ratio of AmB to a pegylated-phospholipid and in some embodiments may contain from about 1:20 to about 1:200 ratio of AmB to pegylated-phospholipid. For example, the present compositions may have a ratio of about 1:20, about 1:40, about 1:60, about 1:80, about 1:90, about 1:100, about 1:120, about 1:140, about 1:160, about 1:180, about 1:200, or a range between and including any two of the foregoing values. In some embodiments, the compositions have a mole to mole ratio of AmB to pegylated-phospholipid of about 1:25 to about 1:100 or even about 1:200. In certain embodiments, the mole ratio of amphotericin B to a pegylated-phospholipid (e.g., PEG-DSPE) is about 1:30 to about 1:100.

In some embodiments the present compositions may include AmB in combination with a second antifungal agent such as 5-fluorocytosine (5-FC). In some embodiments, the concentration of 5-FC is about 0.01 mg/mL, about 0.05 mg/mL, about 0.1 mg/mL, about 0.5 mg/mL, about 1 mg/mL, about 3 mg/mL, about 5 mg/mL, about 7 mg/mL, about 9 mg/mL, about 12 mg/ml, about 15 mg/mL, or about 18 mg/mL, or a range between and including any two of the foregoing values. For example, in some embodiments, the concentration of 5-FC ranges may be from about 1 mg/mL to about 20 mg/mL or about 5 mg/mL to about 15 mg/mL. In one embodiment, the concentration of 5-FC in the present compositions may be about 8 mg/mL, about 9 mg/mL, about 10 mg/mL, or about 11 mg/mL. For example, the concentration of 5-FC may be about 10 mg/mL. In some embodiments, the concentration of 5-FC ranges may be from about 30 μg/mL to about 120 μg/mL. In another embodiment, the concentration of 5-FC in the present compositions is about 50 μg/mL or about 100 μg/mL.

In some embodiments, the present compositions may include micelles with a Z-average particle diameter of less than about 500 nm, less than about 250 nm, less than about 100 nm, less than about 50 nm, or a range between and including any two of the foregoing values. In some embodiments, the micelle particle diameter ranges from about 10 nm to about 150 nm, about 15 nm to about 100 nm, about 16 nm to about 75 nm, about 17 nm to about 50 nm, about 18 to about 40 nm, about 19 to about 35 nm, about 20 to about 30 nm, or a range between and including any two of the foregoing values. For example, in some embodiments, the micelle particle diameter may be from about 22 nm to about 27 nm. In one embodiment, the micelle particle diameter is about 25 nm. In some embodiments the present compositions may include micelles with a polydispersity index (PDI) of less than about 0.5, less than about 0.4, less than about 0.35 nm, less than about 0.3 nm, or a range between and including any two of the foregoing values. In some embodiments, the micelle PDI ranges from about 0.02 to about 0.5, about 0.03 to about 0.4, about 0.04 to about 0.35, about 0.1 to about 0.3, or a range between and including any two of the foregoing values. For example, in some embodiments, the micelle PDI may be from about 0.1 to about 0.35 or about 0.04 to about 0.2 nm.

Typically, the present compositions are stable; i.e., AmB does not precipitate out of solution and/or aggregate after a short period of time. For example, the compositions may be stable for at least about 1 hour, at least about 2 hours, at least about 3 hours, at least about 4 hours, or at least about 5 hours. In some embodiments, the compositions may be stable for up to about 1 week, about 5 days, about 4 days, about 3 days, about 2 days, or about 1 day. In some embodiments, the compositions may be stable for about 1 hour to about 1 week, about 2 hours to about 4 days, about 3 hours to about 3 days, about 4 hours to about 2 days, or a range between and including any two of the foregoing values.

It will be understood that the compositions described herein may include various additives to improve stability or biocompatibility. For example, the compositions may include buffer salts such as phosphate salts, and may also include a pharmaceutically acceptable preservative. Compositions intended for parenteral use, such as for injection will be sterile, having been prepared with sterile water or other sterile aqueous solution. However, the present compositions typically are substantially free of organic solvents, e.g., less than about 2 wt % of ethanol, dimethyl sulfoxide, castor oil, and castor oil derivatives (i.e., polyethoxylated camphor compounds such as Cremophor EL) based on the weight of the composition. In some embodiments the amount of organic solvent is less than about 1 wt %, less than about 0.5 wt %, less than about 0.1 wt %, or free of detectable amounts of organic solvents.

In one embodiment of the present technology, the composition includes a pegylated-phospholipid selected from PEG-DSPE, wherein the PEG block has an average molecular weight of about 3,000 Da to about 7,000 Da; 0.05-0.2 mg/mL amphotericin B; 0.05-0.2 mg/mL deoxycholate sodium salt; wherein the mole ratio of amphotericin B to PEG-DSPE is about 1:30 to 1:100 and the concentration of PEG-DSPE is above its critical micelle concentration. In some embodiments, the composition may further include saline having a concentration of about 0.5 wt % sodium chloride to about 1.5 wt % sodium chloride. In some embodiments, the composition may include about 5 mg/mL 5-FC to about 15 mg/mL 5-FC.

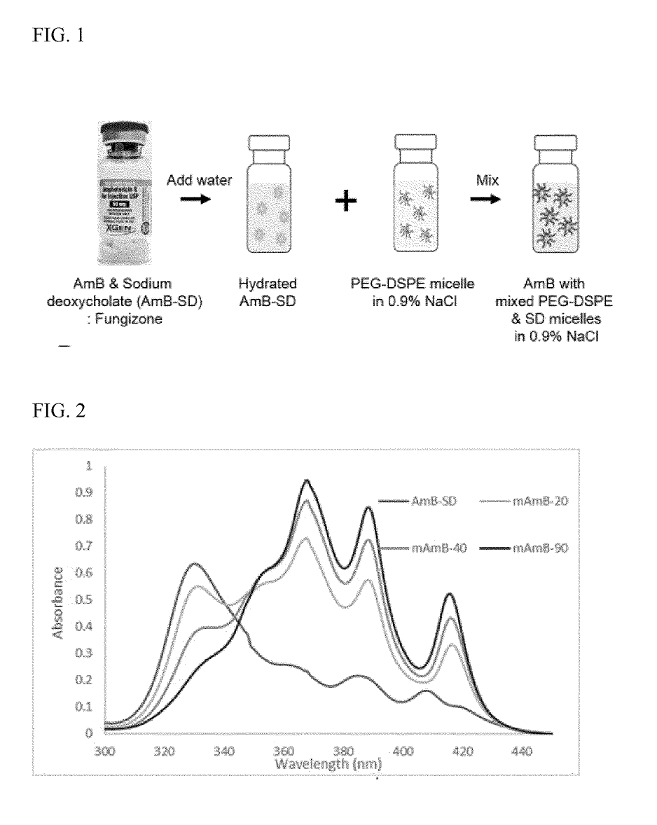

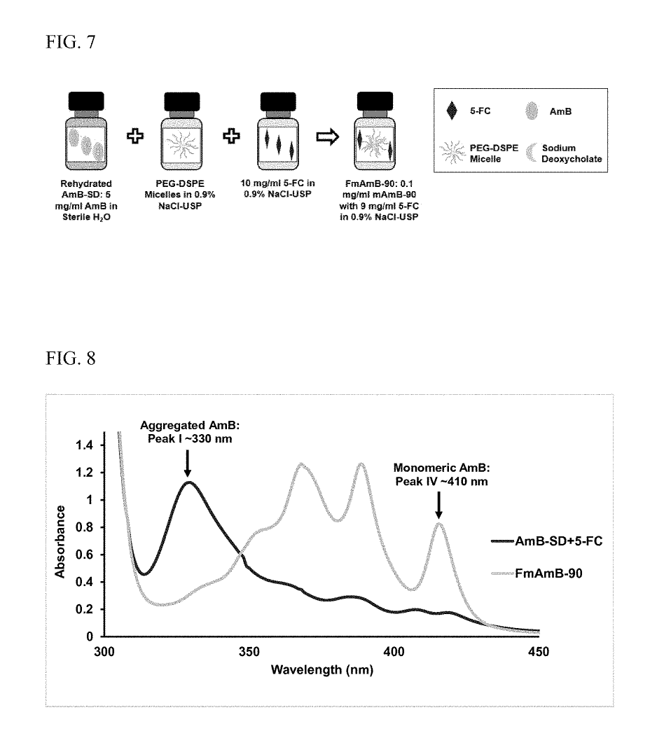

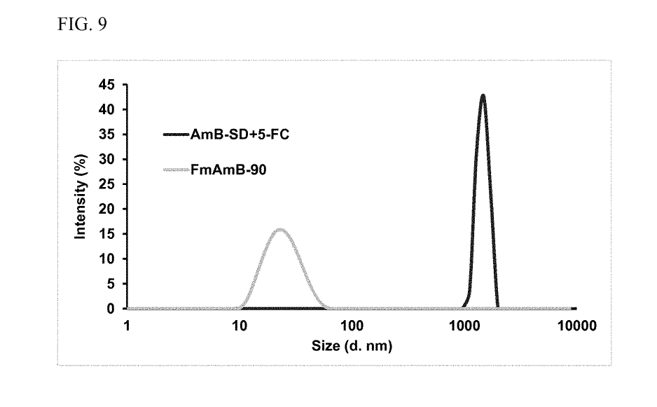

In contrast to complex and expensive multicomponent AmB formulations such as the previously discussed liposomal formulations, the present compositions are simple, readily, and relatively inexpensively prepared. The present compositions may be prepared by mixing an aqueous solution of the pegylated-phospholipid with an aqueous mixture of the amphotericin B and the water soluble salt of the deoxycholate to provide the concentration of pegylated-phospholipid above its critical micelle concentration and the stated mole ratio of amphotericin B to pegylated-phospholipid (as described herein). In some embodiments, the pegylated-phospholipid is prepared in any of the saline solutions described herein. FIG. 1 illustrates schematically the preparation of one embodiment of the present technology using PEG-DSPE. In another embodiment, the peglyated-phospholipid, AmB, the water soluble salt of deoxycholate, and optional saline mixture may be added to an aqueous mixture of 5-FC. In some embodiments, the 5-FC is in any of the saline solutions described herein. FIG. 7 illustrates schematically the preparation of one embodiment of the present technology includes 5-FC.

Thus, in another aspect, the present technology provides kits for reformulating AmB for use parenterally. The kits include a package (e.g., a vial) containing a solid including AmB in admixture with a water soluble deoxycholate salt as described herein, e.g., deoxycholate sodium salt. The amounts of each are sufficient to provide the concentrations of the compositions described herein. For example, the kit could include a package containing about 50 mg of solid AmB in admixture with about 41 mg of deoxycholate sodium salt (such as, but not limited to Fungizone®) and optionally a package containing sterile water for use in preparing a suspension of the AmB. The solid may include additional additives as described herein, e.g., phosphate buffer salts. The kit could also include a package containing an aqueous solution of any of the pegylated phospholipids described herein, e.g., PEG-DSPE. In some embodiments the aqueous solution is saline at a sufficient concentration (e.g., about 0.9 wt % NaCl) to provide any of the final concentrations of the components described herein. In some embodiments, the kit could include a package (e.g., vial) containing 5-FC. For example, the kit could include a package containing about 1000 mL of a solution of 10 mg/mL 5-FC in solution (e.g., about 0.9 wt % NaCl).

In another aspect, the present technology provides methods of treatment that include administering an effective amount of any of the compositions described herein to a mammal suffering from a fungal infection. In some embodiments the mammal is a human. In some embodiments, the mammal is infected by one or more of Candida species, Saccharomyces cerevisiae, Histoplasma capsulatum, Coccidioides immitis, Blastomyces dermatitidis, Rhodotorula, Cryptococcus neoformans, Sporothrix schenckii, Mucor mucedo, and Aspergillus fumigatus. In some embodiments, the one or more of Candida species is an invasive species. In certain embodiments, the mammal is infected by Candida albicans. In some embodiments the mammal is infected by Cryptococcal meningitis.

The compositions described herein can be formulated for various routes of administration, for example, by parenteral, rectal, nasal, vaginal administration, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not interfere with formation of the micelles described herein. Injectable forms may be prepared with acceptable solvents or vehicles including, but not limited to sterilized water, Ringer's solution, 5% dextrose, or an isotonic aqueous saline solution.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

Specific dosages may be adjusted depending on conditions of disease, age, body weight, general health conditions, sex, and diet of the subject, dose intervals, administration routes, excretion rate, and combinations of drug conjugates. Any of the above dosage forms containing effective amounts are well within the bounds of routine experimentation and therefore, well within the scope of the instant present technology. By way of example only, such dosages may be used to administer effective amounts of the compositions to the patient and may include about 0.1 mg/kg, about 0.2 mg/kg, about 0.3 mg/kg, about 0.4 mg/kg, about 0.5 mg/kg, about 0.75 mg/kg, about 1 mg/kg, about 1.25 mg/kg, about 1.5 mg/kg, or a range between and including any two of the forgoing values. Such amounts may be administered parenterally as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once per day, per 2 days, per 3 days, per week, per 10 days, per 2 weeks, or a range between and including any of the foregoing frequencies. Alternatively, the compositions may be administered once per day on 2, 3, 4, 5, 6 or 7 consecutive days. A complete regimen may thus be completed in only a few days or over the course of 1, 2, 3, 4 or more weeks.

The examples herein are provided to illustrate advantages of the present technology and to further assist a person of ordinary skill in the art with preparing or using the micelle compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### Examples

### Example 1: Preparation of AmB-SD with PEG-DSPE Micelles in Saline

Amphotericin B for Injection USP (X-GEN Pharmaceuticals, Horseheads, N.Y.) was rehydrated with 10 mL sterile water for injection without a bacteriostatic agent USP (Baxter, Deerfield, Ill.), according to manufacturer's instructions, resulting in a 5 mg/mL stock solution. PEG(5,000)-DSPE (Avanti Polar Lipids, Alabaster, Ala.) was dissolved in 0.9% NaCl-USP (Baxter, Deerfield, Ill.) to the desired concentration. Three formulations containing AmB-SD and PEG-DSPE micelles in mole to mole ratios of 1:20, 1:40, and 1:90 were analyzed in Examples 2-4. From here on, these formulations will be referred to as mAmB-20, mAmB-40, and mAmB-90, respectively.

### Example 2: Degree of AmB Aggregation—IV/I Ratio Determination

Measuring absorbance of AmB at distinct wavelengths using UV-vis spectroscopy provides insight into its degree of self-aggregation. See J. Barwicz, et al., “Effects of the aggregation state of amphotericin-b on its toxicity to mice,” Antimicrobial Agents and Chemotherapy, 36(10), 2310-5 (1992). Samples were prepared at 0.1 mg/mL, the infusion solution of AmB-SD administered clinically. To evaluate the aggregation state of AmB, samples were diluted 10-fold in 0.9% NaCl-USP in a quartz cuvette with a 1 mm path length (Varian, Palo Alto, Calif.) for UV-visible analysis using a CARY 100 Bio UV-visible spectrophotometer (Varian, Palo Alto, Calif.). Absorbance spectra were recorded from 300-450 nm. The IV/I ratio was determined as the ratio of the fourth (˜409 nm) and the first peak (˜328 nm). Measurements were repeated in triplicate over a period of four hours on three separate occasions using independent stocks of AmB-SD and PEG-DSPE solutions. Samples were stored protected from light at room temperature during the experiment.

### Particle Size Determination—Dynamic Light Scattering

Particle size was determined by dynamic light scattering (DLS) using a ZETASIZER Nano-ZS (Malvern Instruments Inc., Worcestershire, UK) with a He—Ne laser light source (4 mW, 633 nm) and a 173° angle scattered light collection configuration to determine the hydrodynamic diameters of AmB formulations. Samples were prepared at 0.1 mg/mL, diluted 5-fold in 0.9% NaCl-USP, and allowed to equilibrate to 25° C. before DLS analysis. Each sample was subjected to ten measurements repeated in triplicate over a period of four hours. Measurements were taken on three separate occasions using independent stocks of AmB-SD and PEG-DSPE solutions. In order to calculate the Z-average particle diameter and PDI from the Stokes-Einstein equation and correlation function, respectively, the Cumulants analysis method was used to curve fit the correlation function. Samples were stored protected from light at room temperature during the experiment.

### Results

The results show that PEG-DSPE micelles deaggregate AmB-SD. Even in the presence of saline, PEG-DSPE micelles deaggregate AmB-SD over time, and monomer to aggregate ratio measurements reveal that equilibrium may be reached within four hours (data not shown). Addition of PEG-DSPE at increasing mole ratios and the resultant deaggregation of AmB-SD was visualized by UV-vis spectroscopy and evidenced by increased absorbance at ˜409 nm and decreased absorbance at ˜328 nm (FIG. 2). AmB-SD alone was highly aggregated, having an average IV/I ratio of <0.20. Increased absorbance at peak IV and a IV/I ratio approaching 0.80 was observed from mAmB-20, indicating partial deaggregation. More extensive deaggregation of mAmB-40 and m-AmB-90 formulations was evidenced by average IV/I ratios exceeding 3.0 and 5.0, respectively (Table I). As a frame of reference, completely deaggregated AmB in DMSO has a IV/I ratio of approximately 7.

TABLE I
Physical Properties of AmB-SD^{a} with and without micelles

| | | Z-Average Particle | |
|---|---|---|---|
| Formulation | IV/I Ratio^{b} | Diameter (nm)^{c} | PDI^{C} |
| AmB-SD | 0.181 ± 0.041 | 3310 | 0.398 |
| mAmB-20 | 0.785 ± 0014 | 498 | 0.679 |
| mAmB-40 | 3.42 ± 0.31 | 27.2 | 0.312 |
| mAmB-90 | 5.16 ± 1.4 | 23.2 | 0.175 |

^{a}The original concentration of AmB for each formulation was 0.10 mg/mL. Monomer to aggregate ratio, particle size, and PDI values are measurements taken four hours after formulation preparation.
^{b}IV/I ratio presented as mean ± standard deviation (n = 3).
^{c}Mean Z-average particle diameter and PDI presented (n = 3).

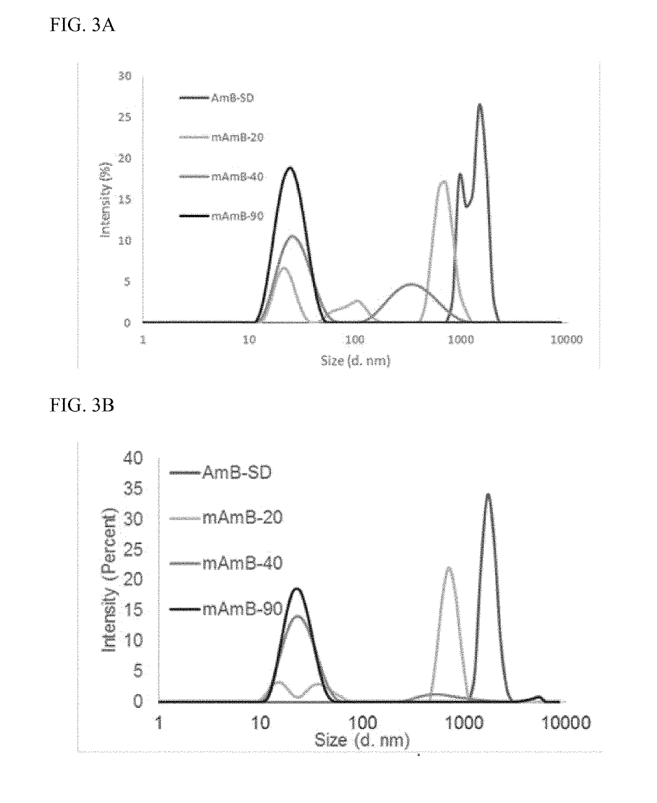

AmB-SD formed large, micron-sized aggregates in 0.9% NaCl-USP. However, addition of PEG-DSPE decreased particle size to nanoscale. Reduced particle size population heterogeneity was observed from mAmB-40 and mAmB-90 formulations (Table I). MAmB-20 and mAmB-40 analysis revealed multimodal size distributions that evolved over a four hour period. MAmB-20 trended towards larger particle size and higher polydispersity while size and polydispersity of mAmB-40 decreased over time. We observed a unimodal size distribution that was maintained over four hours from mAmB-90 (FIGS. 3A and 3B).

### Discussion

Simple addition of PEG-DSPE micelles in saline deaggregated AmB-SD without methods requiring complex protocols or use of specialized equipment. For IV infusion in clinical settings, 5 mg/mL AmB-SD is diluted to 0.1 mg/mL in D5W-USP, resulting in a highly aggregated form of the drug. Immediately after dilution in D5W, particle size is under 100 nm. However, as is known, this size increases over time. When AmB-SD is diluted to 0.1 mg/mL in 0.9% NaCl, AmB remains highly aggregated and particle size is even larger, reaching micron size. Adding increasing amounts of PEG-DSPE micelles reduced the degree of aggregation and decreased particle size (Table I).

The interaction between sodium deoxycholate (SD) and PEG-DSPE was evaluated. The two were combined in mole ratios simulating the different mAmB formulations. A single peak was evident after DLS analysis, suggesting that SD and PEG-DSPE form mixed micelles (data not shown). Deaggregation may be achieved from disruption of AmB aggregates by formation of mixed PEG-DSPE and SD micelles followed by partitioning of monomeric AmB into the mixed micelle core (FIGS. 3A and 3B).

### Example 3: Hemolysis Assay and Renal Toxicity of AmB-SD with and without Micelles

### Hemolysis Assay

To determine the hemolytic activity of different formulations, bovine red blood cells (Innovative Research, Novi, Mich.) were washed three times in 1×PBS (Corning cellgro, Manassas, Va.), diluted 200-fold in PBS, and further diluted in a 1:1 (v/v) ratio with AmB-SD alone or in the presence of PEG-DSPE micelles. Samples were incubated at 37° C., shaking at approximately 200 rpm. After 30 minutes of incubation, samples were placed on ice for 5 minutes to halt hemolysis, then centrifuged at 13,000 rpm for 30 seconds. Supernatant was transferred to a 96-well plate and analyzed for absorbance at 405 nm. The negative control consisted of cells in a 1:1 dilution with 1X PBS. The positive control sample, in which total lysis (TL) of cells was achieved, was prepared with cells in the presence of 25 μg/mL AmB-SD in PBS. In addition to different AmB concentrations, the hemolytic activity of PEG-DSPE alone was tested. Experiments were performed in triplicate. Percent hemolysis was calculated using the following equation in which PBS and TL represent the negative and positive controls, respectively: Percent Hemolysis=((Sample−PB S)/(TL−PBS))*100%.

### Renal Toxicity

An infusion study was performed to determine the renal toxicity of the different AmB formulations after a three day dosing regimen in rats. Jugular vein-cannulated male Sprague-Dawley rats (250-300 kg) were purchased from Charles River (Raleigh, N.C.). Animals were housed individually to avoid displacement of cannula, and had free access to water and rat chow in a temperature and light-controlled room for at least one week before the first infusion. The cannulated rats were divided into three groups: AmB-SD in D5W-USP (Fun), mAmB-90 (M), and mAmB-90 in D5W-USP (M-D). Using rodent infusion kits (SAI Technologies, Lake Vila, Ill.), each rat was connected to an Elite Infusion Pump 11 (Harvard apparatus, Holliston, Mass.). Each group received a daily 1.5-hour infusion of 2 mg/kg AmB for three days. The total volume of each infusion was 7.5 mL. Before beginning the study and 24 hours after the final infusion, approximately 1.5 mL blood was collected from cannula and transferred into heparinized tubes (BD Vacutainer®, Franklin Lakes, N.J.). Whole blood samples were centrifuged at 2000 rpm for 10 minutes to allow for separation and collection of plasma. Plasma samples were immediately taken to UW-Madison's Veterinary Care Center for kidney toxicity analysis, where levels of creatinine and blood urea nitrogen (BUN) were determined. In accordance with the US Public Health Service Policy on Human Care and Use of Laboratory Animals, all animal handling and care procedures complied with a University of Wisconsin-Madison Institutional Animal Care and Use Committee (IACUC) approved protocol.

### Results

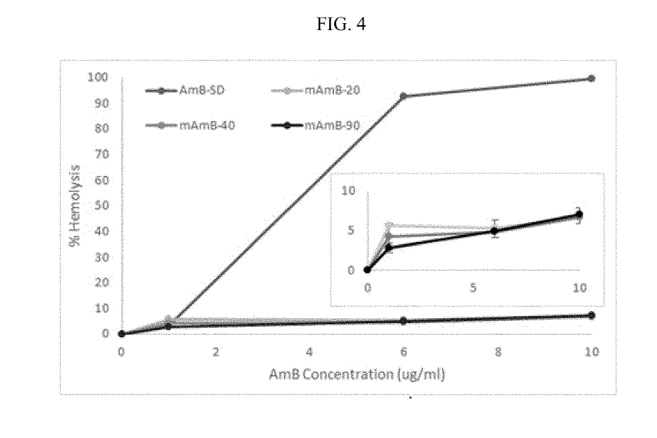

Hemolytic activity was used to assess in vitro mammalian cell membrane toxicity of AmB-SD and mAmB formulations using bovine red blood cells as a model. MAmB-20, mAmB-40, and mAmB-90 did not exceed 10% hemolysis at any tested AmB concentration (FIG. 4, Inset). AmB-SD was highly hemolytic, reaching nearly complete hemolysis at concentrations as low as 2 μg/mL (FIG. 4). Above 1 μg/mL, AmB-SD was significantly more hemolytic than each mAmB formulation (P<0.0001). PEG-DSPE alone exhibited negligible hemolytic activity (<1%).

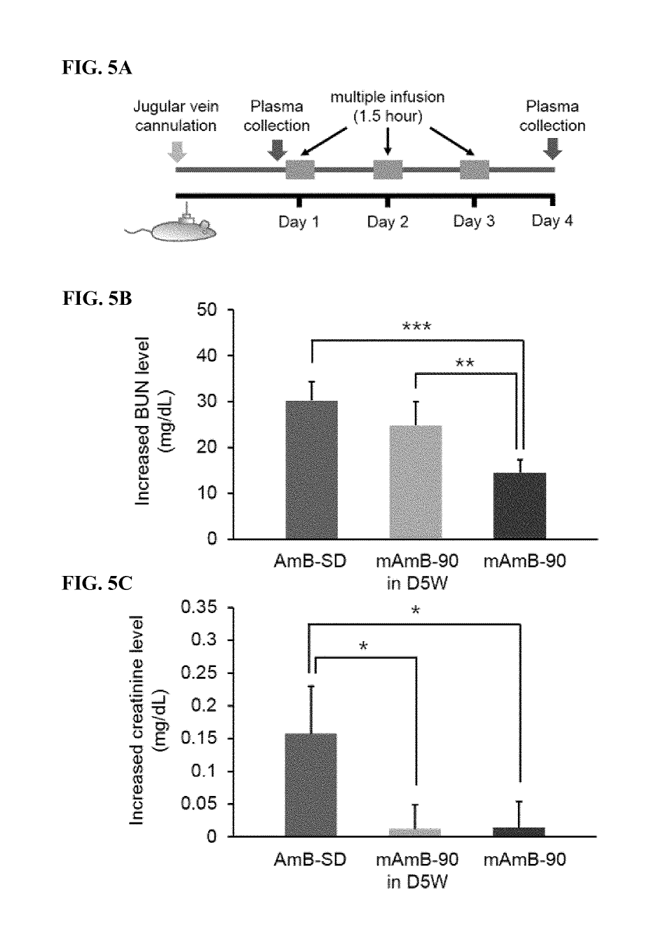

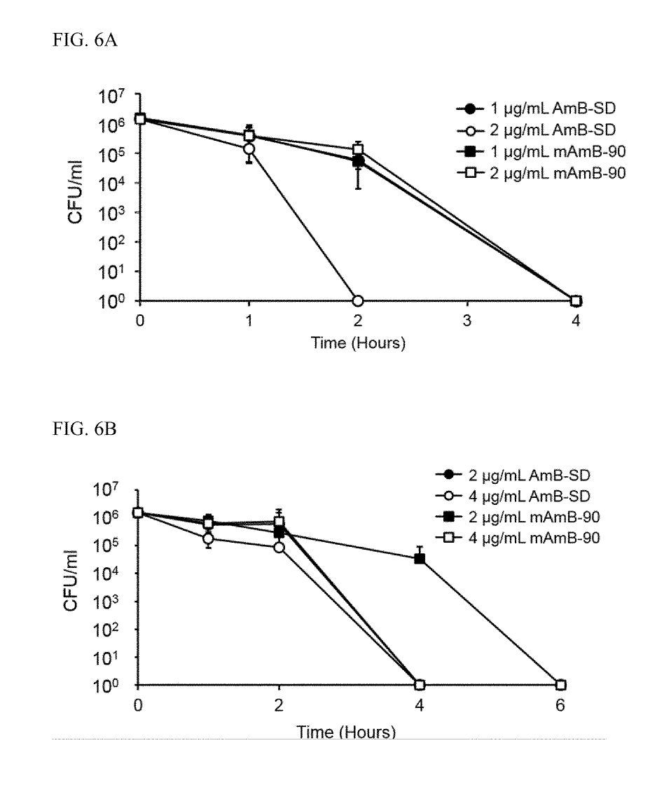

Increases in BUN and creatinine after daily infusions of 2 mg/kg AmB as AmB-SD in D5W, mAmB-90 in D5W, or mAmB-90 (in saline) over three days were monitored to determine renal toxicity. While mAmB-90 and the other mAmB formulations discussed previously were prepared with saline, mAmB-90 prepared with D5W was included in this experiment to determine whether or not saline plays a role in reducing renal toxicity. Results are shown in FIG. 5. Respectively, BUN increases following treatment with AmB-SD and mAmB-90 in D5W were approximately 2-fold and 1.5-fold greater than BUN levels following mAmB-90 treatment. MAmB-90 in D5W caused considerably higher levels of BUN than mAmB-90 (interpreted as significant (P<0.01)). The increase in creatinine following AmB-SD treatment was over 10-fold greater than the creatinine increase observed after mAmB-90 in D5W and mAmB-90 treatment. Average creatinine increases following mAmB-90 in D5W or mAmB-90 treatment were similar and quite low at less than 0.015 mg/dL.

### Discussion

### In Vitro Toxicity

The extensive hemolytic activity of AmB above its critical aggregation concentration (CAC), approximately 1 μg/mL, is known. The present mAmB formulations, although deaggregated to different extents, exhibited significantly reduced hemolytic activity compared to AmB-SD alone, which was highly hemolytic at concentrations as low as 2 μg/mL (FIG. 4). Although mAmB-20 exhibited strong absorbance at the characteristic aggregation wavelength (FIG. 2), its hemolytic activity was similar to the other less aggregated formulations (FIG. 4). This suggests that both deaggregation of AmB and the interaction of the PEG-DSPE with the surrounding environment may all contribute to the significant reduction in hemolytic activity of the PEG-DSPE formulations.

### In Vivo Toxicity

BUN and creatinine levels, markers of renal function, were significantly greater in the AmB-SD treated group than in both mAmB-90 in D5W and mAmB-90 groups. This suggests that renal function is damaged by aggregated AmB but not monomeric AmB. Interestingly, BUN in rats treated with mAmB-90 in D5W was significantly greater than BUN in mAmB-90 treated rats. This reinforces previous studies' reports on the protective effect of saline on renal function. However, there was only a negligible difference in increased creatinine between mAmB-90 in D5W and mAmB-90 treated groups.

### Example 4: Antifungal Efficacy of AmB with and without Micelles

### In Vivo Antifungal Efficacy

### Minimum Inhibitory Concentration (MIC)

The MIC was determined as the lowest concentration of AmB in a given formulation that considerably inhibited visible fungal growth of S. cerevisiae strain 9763 (American Type Culture Collection, Manassas, Va.) or C. albicans strain K1 colonies. To prepare the inoculation, the fungal strains were cultured for approximately 24 hours on Sabouraud dextrose agar (SDA) plates were diluted to a 0.5 McFarland standard (1-5×10⁶ CFU/mL) in sterile normal saline and diluted 100-fold in yeast peptone dextrose (YPD). Inoculum was added to serial dilutions of AmB in 96-well plates, following the microplate dilution method. Plates were sealed with Breathe Easier membranes (Sigma Aldrich, St. Louis, Mo.). ATCC 9763 or K1 was incubated at 30° C. or 37° C., respectively, for 18-20 hours. Experiments were repeated in triplicate on three separate occasions with distinct ATCC 9763 or K1 colonies and independent stocks of AmB-SD and PEG-DSPE. SDA was prepared with 1% peptone, 4% glucose, and 1.5% agar in deionized water. YPD media was prepared with 1% yeast extract, 2% peptone, and 2% glucose in deionized water. Autoclaving was used to sterilize media.

### Minimum Fungicidal Concentration (MFC)

The MFC was interpreted as the lowest concentration of drug that inhibited ≧95% fungal growth. From the MIC experiment, 50 μl of each concentration above the MIC was plated in triplicate on a new 96-well plate with wells filled with 150 μl YPD. Plates were sealed as before and incubated for approximately 24 hours. Results were read by eye and turbidometrically with the Spectramax M2 (Molecular Devices, Sunnyvale, Calif.) set to detect absorbance at 580 nm. Experiments were repeated in triplicate with independent colonies and stocks of AmB-SD and PEG-DSPE.

### Time Kill Assay

Time kill assays were used to determine the antifungal activity of different formulations at various AmB concentrations relative to one another over time. ATCC 9763 or K1 colonies were diluted to a 0.5 McFarland standard in sterile normal saline, diluted 1000-fold in YPD, and added to AmB formulations in sterile test tubes. Samples were incubated at 30° C. or 37° C., shaking at approximately 200 rpm. At predetermined timepoints, 100 μl sample was removed, diluted five-fold in 0.9% NaCl, and spread upon an SDA plate. Colonies were counted after 48 hours of incubation. Again, experiments were repeated in triplicate with independent colonies and stocks of AmB-SD and PEG-DSPE.

### Results

The MICs and NIFCs of AmB-SD alone or in the presence of PEG-DSPE micelles dissolved in 0.9% NaCl-USP were maintained across the different formulations (Table II). In ATCC 9763 time kill studies (FIG. 6A) at 1 μg/mL AmB, AmB-SD and mAmB-90 resulted in complete absence of CFU after four hours. At 2 μg/mL AmB, no CFUs were evident after two hours of treatment with AmB-SD, while mAmB-90 treatment reduced the CFU count to zero after four hours. The MIC of AmB-SD and mAmB-90 against K1 ranged from 0.25-0.5 μg/mL, and the MFC ranged from 0.5-1 μg/mL. K1 time kill studies (FIG. 6B) in which 4 and 2 μg/mL AmB as AmB-SD and 4 μg/mL AmB as mAmB-90 decreased CFU counts to zero within four hours. MAmB-90 at 2 μg/mL AmB resulted in absence of CFUs between four and six hours.

TABLE II
Antifungal Activity against S. cerevisiae
of AmB-SD^{a} with and without micelles

| Formulation | MIC (μg/mL) | MFC (μg/mL) |
|---|---|---|
| AmB-SD | 0.25 | 0.5-1 |
| mAmB-20 | 0.25 | 0.5-1 |
| mAmB-40 | 0.25 | 0.5-1 |
| mAmB-90 | 0.25 | 0.5-1 |

^{a}The original concentration of AmB for each formulation was 0.10 mg/mL. Monomer to aggregate ratio, particle size, and PDI values are measurements taken four hours after formulation preparation.

### Discussion

Addition of PEG-DSPE micelles did not affect AmB-SD's fungicidal activity in long-term endpoint assays, i.e. MIC and MFC experiments. Compared to AmB-SD, the present formulations' maintenance of in vitro antifungal activity appears superior to the reduced potency of AmBisome, a liposomal AmB preparation that is used clinically. See E. M. Johnson, et al., “Comparison of in vitro antifungal activities of free and liposome-encapsulated nystatin with those of four amphotericin B formulations,” Antimicrobial Agents and Chemotherapy, 42(6), 1412-6 (1998). In dynamic dose-response time-kill assays, fungal activity was comparable between AmB-SD and mAmB-90 formulations. However, a longer time was required for 2 μg/mL AmB in mAmB-90 to reduce the CFU count to zero relative to AmB-SD at the same concentration (FIG. 6), possibly due to slower release of AmB from the micelles.

### Example 5: AmB-SD+5-FC with Micelles (FmAmB)

Preparation of AmB-SD+5-FC with PEG-DSPE Micelles in Saline (FmAmB-90)

Similar to Example 1, AmB-SD in a 1:90 molar ratio with PEG-DSPE micelles in saline (mAmB-90) was prepared (final AmB level of 1 mg/mL). The mAmB-90 composition was then mixed with 5-fluorocytosine (5-FC) dissolved in saline (10 mg/mL) (FIG. 7). The combination is herein referred to as FmAmB-90 (0.1 mg/mL AmB, 9.0 mg/mL 5-FC, and 2.0 mg/mL PEG-DSPE).

### Degree of Aggregation—IV/I Ratio Determination/Particle Size Determination—Dynamic Light Scattering

Following the procedures of Example 2, the aggregation state and particle size of FmAmB-90 were evaluated with UV-visible spectroscopy and dynamic light scattering, respectively. The IV/I ratio was determined as the ratio of the fourth (˜410 nm) and the first peak (˜330 nm).

### Short-Term Stability

The degree of aggregation and particle size of FmAmB-90 was monitored over 48 hours. Using the methods described above, the monomer to aggregate ratio and particle size of FmAmB-90 were evaluated upon preparation and 1, 24, and 48 hours after preparation. Samples were stored in the dark at room temperature (˜25° C.) during the experiment. The experiment was performed in triplicate.

### Hemolysis Assay

Following the procedures of Example 3, a hemolysis assay was completed to determine the effect of 5-FC on in vitro toxicity of AmB-SD and mAmB-90. The hemolytic activity of 1, 6, and 10 μg/mL AmB as AmB-SD or mAmB-90 was tested in the presence of 50 or 100 μg/mL of 5-FC. These concentrations were chosen because they represent a clinically relevant range that could reasonably be detected in a patient undergoing 5-FC therapy. The hemolytic activity of 5-FC alone was also measured.

### Results

### Degree of Aggregation—IV/I Ratio Determination

FIG. 8. illustrates the absorbance spectra of AmB-SD+5-FC and FmAmB-90 upon preparation. At each time point, the average monomer to aggregate ratio of AmB-SD combined with 5-FC was less than 0.3, indicating a highly aggregated state (data not shown). Compared to the IV/I ratio of AmB-SD alone which was less than 0.20 (Example 2), the formulations with 9 mg/mL 5-FC had a slightly less aggregated state.

TABLE III
Physical Properties of AmB-SD +
5-FC with micelles (FmAmB-90)

| | Monomer to | Z-Average Particle | |
|---|---|---|---|
| Time (h) | Aggregate Ratio | Diameter (nm) | PDI |
| 0 | 3.3 ± 0.064 | 25.3 ± 0.6 | 0.088 |
| 1 | 3.7 ± 0.066 | 23.3 ± 0.2 | 0.049 |
| 24 | 4.0 ± 0.16 | 24.3 ± 0.9 | 0.15 |
| 48 | 3.9 ± 0.069 | 22.9 ± 0.3 | 0.080 |

The UV-visible spectroscopy and dynamic light scattering revealed deaggregation of AmB-SD and stable particle size of FmAmB-90 in 0.9% NaCl-USP, respectively, for up to 96 hours (Table III). At preparation, the monomer to aggregate ratio of FmAmB-90 was roughly 3.3. After one hour, the monomer to aggregate ratio rose to 3.7. A similar degree of aggregation was observed at 24 and 48 hours. These data are consistent with results from the evaluations of mAmB-90 alone (Example 2).

### Particle Size Determination—Dynamic Light Scattering

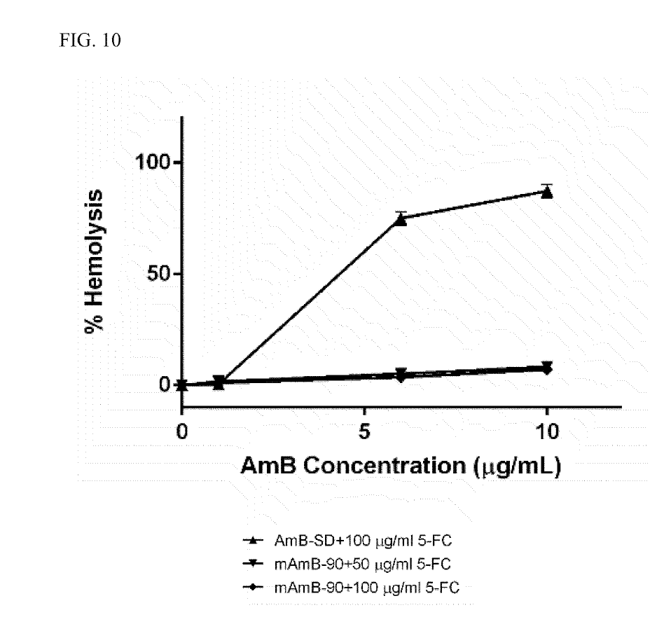

FIG. 9 provides the comparative particle sizes of AmB-SD+5-FC and FmAmB-90. At preparation, AmB-SD combined with 5-FC formed micron-sized particles (˜3 μm). Addition of PEG-DSPE micelles markedly reduced particle size. The particle size of FmAmB-90 was approximately 25 nm throughout the course of 48 hours (Table III). The PDI did not exceed 0.15 at any observed timepoint throughout the stability study's duration, indicating a narrow size distribution.

### Hemolysis Assay

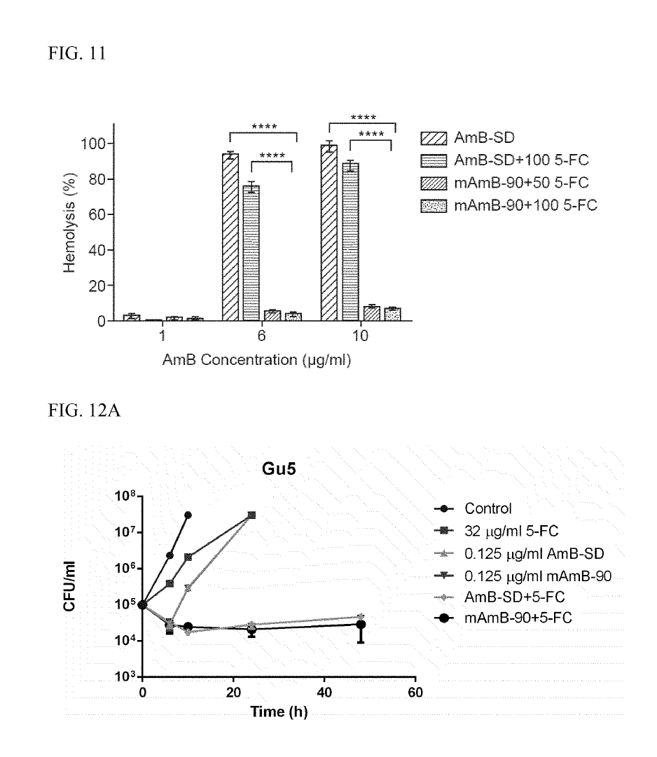

As demonstrated in Example 3, mAmB-90 is significantly less hemolytic compared to AmB-SD. At the tested concentrations, addition of 5-FC did not impact hemolysis (FIG. 10). The concentration range of 5-FC from 25-100 μg/ml exhibited negligible hemolytic activity of <0.4% (FIG. 10 provides data for 50 and 100 μg/mL 5-FC). The hemolytic activity of AmB-SD, AmB-SD+5-FC, and FmAmB-90 (50 and 100 μg/mL 5-FC) are shown in FIG. 11. AmB-SD+5-FC exhibited near total hemolysis at 6 and 10 μg/mL AmB in the presence of 100 μg/mL 5-FC. Although highly hemolytic, this was significantly less active than AmB-SD alone (p<0.0001). Hemolytic activity did not exceed 10% at any tested concentration of FmAmB-90 when 50 and 100 μg/mL of 5-FC was combined with 1, 6, and 10 μg/mL of AmB. The hemolytic activity of FmAmB-90 is comparable to that of mAmB-90 in the same concentration range (Example 2). At 6 and 10 μg/mL, AmB-SD with 100 μg/mL 5-FC exhibited at least 10 times the hemolytic activity measured from either combination of FmAmB-90 (p<0.0001).

### Discussion

### Short-Term Stability

PEG-DSPE micelles deaggregated AmB-SD in the presence of saline and 5-FC to a similar degree of mAmB-90 without 5-FC (average monomer to aggregate ratio of approximately 3.5). Thus, 5-FC addition did not impact the ability of PEG-DSPE micelles to deaggregate AmB-SD. Interestingly, FmAmB-90 continued to deaggregate over time. It is speculated that PEG-DSPE micelles interact with soluble aggregates of AmB-SD, forming mixed micelles with sodium deoxycholate that sequester monomeric AmB to the micelle core containing DSPE. As 5-FC is highly hydrophilic and has a low molecular weight (129 g/mol), it is unlikely that the drug disrupts this process. Rather, 5-FC is most likely outside the micelles or perhaps interacts with the micelle corona containing the hydrophilic PEG block and polar region of sodium deoxycholate. Further supporting our assertion that 5-FC interacts largely with the hydrophilic regions of the system, addition of 5-FC did not impact the size of mAmB-90. An identical study was attempted at the elevated temperature of 45° C. (data not shown). After only one hour, FmAmB-90 became highly aggregated, with an average monomer to aggregate ratio of <0.9. Strengthened hydrophobic interaction at this high temperature may explain the return to the aggregated state.

### In Vitro Hemolysis Assay

Addition of 5-FC did not alter the hemolytic profiles of mAmB-90 at 1, 6, or 10 μg/mL AmB. However, reduced hemolytic activity was noted when 100 μg/mL 5-FC was added to AmB-SD, possibly reflecting precipitated AmB that is non-hemolytic. The hemolytic activity of mAmB-90 alone was comparable to that combined with 50 and 100 μg/mL 5-FC.

### Example 6: Antifungal Efficacy of AmB-SD+5-FC with and without Micelles

In Vitro Antifungal Efficacy Minimum inhibitory concentration (MIC)

Following the procedures of Example 4, the lowest concentration of AmB or 5-FC in a given formulation that considerably inhibited visible fungal growth of three strains of C. albicans (K1, SC5314 (ATCC® MYA-2876), and Gu5 (ATCC® MYA-574)) was determined. Treatment with AmB results in obvious MICs, but 5-FC results in trailing growth that can span a large range of concentrations. For 5-FC, the concentration necessary to inhibit ≧50% as determined by absorbance is reported.

### Checkboard Method

The drug relationship between 5-FC and either AmB-SD or mAmB-90 was evaluated using the fractional inhibitory concentration index (FICI) to compare the in vitro antifungal efficacy of each combination therapy against S. cerevisiae ATCC 9763 and the three strains of C. albicans. K1 was originally isolated from the bloodstream of a patient that was suffering from disseminated candidiasis. Strains SC5314 and Gu5 are known fluconazole resistant strains.

To define the drug interaction between 5-FC combined with either AmB-SD or mAmB-90, the checkerboard method was performed in YPD with a 96-well plate as described by R. L. White et al., “Comparison of three different in vitro methods of detecting synergy: Time-kill, checkerboard, and E test,” Antimicrobial Agents and Chemotherapy, 40(8),1914-8 (1996). Columns 1 and 2 were reserved to determine the MIC of AmB and 5-FC alone, respectively. Column 3 was reserved for the media control and the growth control was plated in column 4. Either AmB-SD or mAmB-90 was serially diluted two-fold from column 5 to 12. From a separate 96-well plate containing serially diluted 5-FC, appropriate dilutions of the drug were added to rows A through H. Inoculum was prepared as described in Example 4 and added to the drug plate. This resulted in concentration ranges of 2 to 256 μg/mL 5-FC and 1.56*10⁻² to 2 μg/mL AmB. The fractional inhibitory concentration index (FICI) was used to analyze results of the experiment. FICI was calculated as follows:

$$FICI = FIC ( X ) + FIC ( Y )$$

$$where FIC ( X ) = \frac{X}{MIC ( X )} and$$

$$FIC ( Y ) = \frac{Y}{MIC ( Y )}$$

FICI values were interpreted as described by Arikan, et al.: FICI≦0.5, synergistic; 0.5<FICI≦1, synergistic to additive; 1<FICI≦4, indifferent; and FICI>4, antagonistic. S. Arikan, et al., “In vitro synergy of caspofungin and amphotericin B against Aspergillus and Fusarium spp,” Antimicrobial Agents and Chemotherapy, 46(1), 245-7 (2002). After 24 hours of incubation, the average FIC score was calculated from the concentration of drugs present in a well containing considerably inhibited fungal growth. The absence of growth was determined by eye, and absorbance at 580 nm was used to detect wells in which fungal growth was reduced by ≧90%. Experiments were repeated on three separate occasions with distinct 24 hour C. albicans colonies and independent stocks of AmB-SD, 5-FC, and PEG-DSPE. Reported values represent the average of results detected by eye.

### Time Kill Assay

In addition to the checkerboard method, time-kill assays were used to define the relationship between 5-FC and AmB-SD with and without micelles. Colonies were adjusted to 1 to 5×10⁶ CFU/mL in sterile normal saline. The yeast was diluted 10-fold in YPD alone or containing drug. Samples in 5 mL test tubes were incubated at 37° C. shaking at a speed of 200 rpm. At 0, 6, 10, 24, and 48 hours, samples was serially diluted 10-fold in sterile normal NaCl, and 20 μL of each dilution was plated on SDA. In cases where less than 12,500 CFU/mL was expected (≦25 CFU from the 10-fold dilution), 20 μL was plated directly from the sample tube. The theoretical limit of quantitation for these practices is 1,250 CFU/mL. Colonies were counted after 24 to 48 hours of incubation. Those with CFUs in the countable range as recommended by the United States Pharmacopeia (25-250 CFUs) were included in the dataset. Experiments were performed in duplicate on two separate occasions and the average CFU/mL was calculated for each replicate. Drug interactions were defined as previously described in E. Canton, et al.: a ≧2 log₁₀ decrease in CFU per milliliter for a combination compared to CFU reduction of the most active drug alone as synergy, a <2 log₁₀ increase or decrease in CFU as indifference, and an increase of ≧2 log₁₀ as antagonism. E. Canton, et al., “Synergistic activities of fluconazole and voriconazole with terbinafine against four Candida species determined by checkerboard, time-kill, and Etest methods,” Antimicrobial Agents and Chemotherapy, 49(4),1593-6 (2005).

### In Vivo Antifungal Efficacy

The activity of each drug, alone and in combination, was evaluated in a neutropenic mouse model of disseminated candidiasis as described previously by D. Andes, et al., “Pharmacodynamics of amphotericin B in a neutropenic-mouse disseminated-candidiasis model,” Antimicrobial Agents and Chemotherapy, 45(3), 922-6 (2001). Treatment groups were as follows: control (no drug), 5-FC (72 mg/kg), AmB-SD (2 mg/kg), mAmB-90 (2 mg/kg), AmB-SD+5-FC (2 mg/kg+72 mg/kg), and mAmB-90+5-FC (2 mg/kg AmB-SD+72 mg/kg 5-FC).

For each treatment group, three six-week-old ICR/Swiss specific-pathogen-free female mice (Harlan Sprague Dawley, Madison, Wis.) were rendered neutropenic by multiple doses of cyclophosphamide. Animals were classified as neutropenic when polymorphonuclear leukocyte counts dropped below 100/mm³. Infection was established by injecting 100 μL inoculum into the lateral tail vein. The inoculum was prepared by subculturing K1 on SDA plates for 24 hours at 35° C. Six colonies were dispersed into 5 mL of sterile pyrogen-free normal saline warmed to 35° C. and injected two hours prior to drug treatment. Antifungals (200 μL) were dosed intraperitoneally on the day of infection and 24 hours after the initial dose. Because 5-FC causes AmB-SD to precipitate, two separate 200 μL injections were administered to the AmB-SD+5-FC group. The animals were euthanized by CO₂ asphyxiation 48 hours after the initial drug treatment. Their kidneys were immediately harvested and homogenized in 4° C. sterile saline. Kidney homogenate was diluted 10-fold serially and aliquots were plated on SDA. After 24 hours of incubation at 35° C., colonies were counted. Results were expressed as the mean and standard deviation of the difference in viable fungal counts between the control group at zero hour (two hours post-infection) and each group at the 48 hour timepoint. The data represents three mice (six kidneys). Statistical differences between groups were compared using one-way ANOVA with Tukey's multiple comparisons test (GraphPad Prism 6, La Jolla, Calif.). Statistical significance was acknowledged when p-values were less than or equal to 0.05.

### Results

### In Vitro Antifungal Efficacy

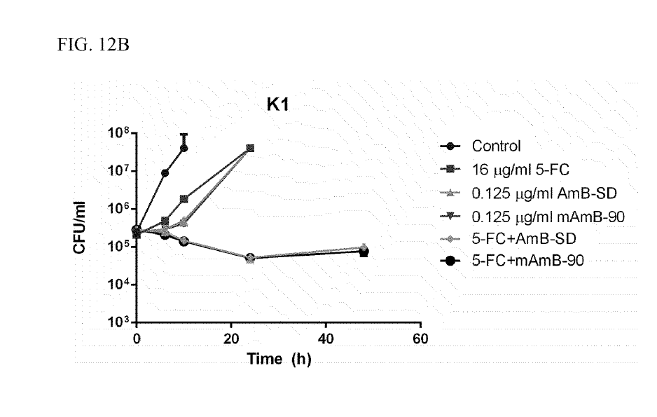

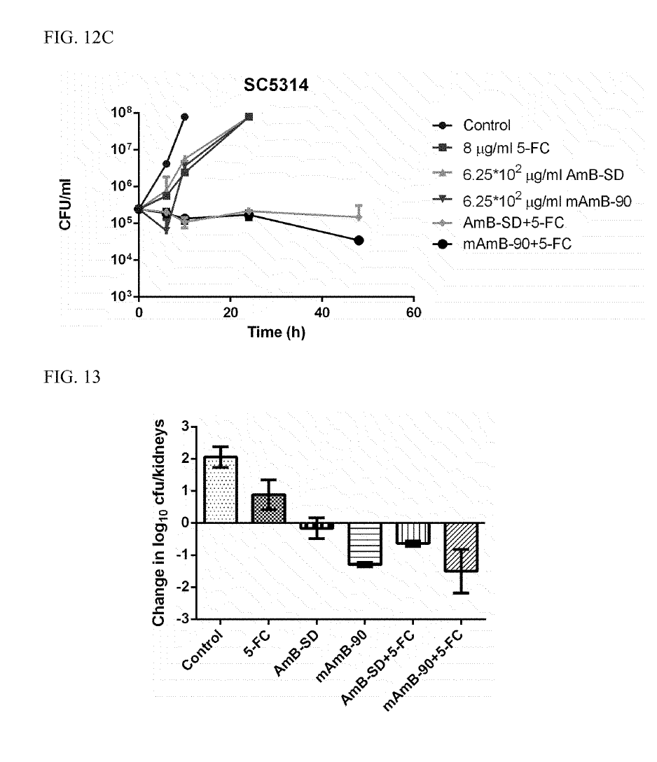

As provided in Table IV, the addition of PEG-DSPE micelles to AmB-SD did not alter its fungicidal efficacy in MIC. As illustrated in FIGS. 12A, 12B, and 12C, the time-kill experiments with Gu5 (12A), K1 (12B), and SC5314 (12C) showed a synergistic relationship between AmB+5-FC with and without micelles. This trend for each C. albicans strain, suggests usefulness of the combination against fluconazole-resistant strains such as SC5314 and Gu5. In each case, the growth control was turbid with growth at 10 hours. At 0.25 and 0.5 times the MIC of 5-FC and AmB, each single-drug treated sample was turbid with growth at 24 hours (data not shown). Combination treatment with drug concentrations equal to one-fourth of the highest documented MIC greatly reduced fungal burden.

TABLE IV
MIC for 5-FC and AmB-SD with and without micelles MIC

| Organism | 5-FC | AmB-SD | mAmB-90 |
|---|---|---|---|
| Gu5 | 81 | 0.35 | 0.35 |
| K1 | 64 | 0.31 | 0.31 |
| SC5314 | 20 | 0.25 | 0.25 |

As provided in Table V, the addition of micelles to AmB-SD+FC did not alter fungicidal efficacy in checkerboard experiments. Additionally, the FICI values ranged from 0.5-0.8, which confirms an additive to synergistic drug interaction between AmB and 5-FC.

TABLE V
FICI for AmB-SD + 5-FC with and without micelles
FICI^{a}

| Organism | 5-FC + AmB-SD | 5-FC + mAmB-90 |
|---|---|---|
| Gu5 | 0.77 | 0.75 |
| K1 | 0.56 | 0.54 |
| SC5314 | 0.56 | 0.56 |

^{a}FICI Interpretation: Synergistic (<0.5); Synergistic to Additive (0.5-1); Indifferent (1-4); Antagonistic (>4).

### In Vivo Antifungal Efficacy

As illustrated in FIG. 13, each treatment formulation resulted in a significantly lower fungal burden compared to the control. The weakest effect was noted in the group that received 72 mg/kg 5-FC alone. At 48 hours, the fungal burden of the control group was 1.2 times that of the 5-FC treated group (p=0.027). The greatest reduction in fungal burden was caused by treatment with mAmB-90+5-FC (i.e., FmAmB-90), which had a mean difference from the control of approximately 3.56 log₁₀ CFUs (p<0.0001). The second most effective formulation was provide by mAmB-90 with a mean difference of 3.35 log₁₀ CFUs (p<0.0001). As such, treatment with mAmB-90 was not significantly different from combination therapy using our reformulation, mAmB-90 (p=0.98). Similarly, treatment with AmB-SD+5-FC did not result in a significantly different fungal burden relative to treatment with AmB-SD alone (p=0.67). Using the aforementioned guide for determining drug interactions, the relationship between AmB and 5-FC was defined as indifferent at the tested concentrations, as the difference in fungal burden between the most active drug (AmB) and combination treatment was less than 2 log₁₀ CFU. However, FmAmB-90 was more effective than AmB-SD monotherapy. Viable colonies after AmB-SD therapy exceeded those detected after FmAmB-90 treatment by a factor of 1.5 (p=0.011). Interestingly, mAmB-90 was significantly more effective than AmB-SD at the same dose of 2 mg/kg. At the 48 hour endpoint, the fungal burden of AmB-SD was 1.4 times that of mAmB-90 (p=0.034). Treatment with AmB-SD+5-FC resulted in a fungal burden 1.3 times that following FmAmB-90 treatment. Treatment with FmAmB-90 reduced fungal burden to 2 log₁₀ CFUs, the lowest detected colony count in the data set. However, data collected from the FmAmB-90 treated group was the most variable, with the highest range in the dataset of 1.3 log₁₀ CFUs. Treatment with FmAmB-90 was not significantly different from AmB-SD+5-FC (p=0.14).

### Discussion

### In Vitro Antifungal Efficacy

Addition of PEG-DSPE micelles did not affect the antifungal activity of AmB-SD alone or in combination with 5-FC in in vitro experiments. No differences in activity were seen in long-term 24 hour end-point assays (i.e. MIC and the checkerboard experiment). Although the mean fungal burden is consistently lower after treatment with mAmB-90 in time-kill experiments with 48 hours of drug exposure, the difference in fungicidal activity between mAmB-90 and AmB-SD is not significant in most cases.

### In Vivo Antifungal Efficacy

The static activity of AmB-SD in vivo against K1 is consistent with previous findings. D. Andes, et al., “Pharmacodynamics of amphotericin B in a neutropenic-mouse disseminated-candidiasis model,” Antimicrobial Agents and Chemotherapy, 45(3), 922-6 (2001). Andes et al. studied the relationship of dosing intervals to efficacy and determined the fungistatic dose range associated with a 24 hour dosing interval was 2.01 to 2.79 mg/kg AmB-SD. The enhanced activity of mAmB-90 in vivo compared to AmB-SD is surprising. It is speculated that the enhanced activity may be explained by the aggregation state hypothesis. That is, the enhanced potency of deaggregated AmB may stem from a contribution by one or more of the following: 1. monomeric AmB is selective for ergosterol, meaning it avoids nonspecific interactions with mammalian cells; 2. monomeric AmB does not heavily associate with itself, potentially leading to a higher effective concentration as more drug molecules are available to bind to ergosterol; and 3. monomeric AmB interacts with serum components differently from the aggregated form.

Deaggregation of AmB and a particle size of less than 30 nm were maintained in the FmAmB formulations (see Table III). Accordingly, FmAmB can be used to simultaneously IV deliver monomeric AmB, 5-FC, and sodium supplementation with a reduced risk of combination therapy toxicity. This simultaneous IV delivery can provide antifungal efficacy, ease of preparation, and commercial availability of formulation components.

### EQUIVALENTS

While certain embodiments have been illustrated and described, a person with ordinary skill in the art, after reading the foregoing specification, can affect changes, substitutions of equivalents and other types of alterations to the conjugates and micelles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds, or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. A composition comprising:
pegylated-phospholipid,
amphotericin B,
a water-soluble deoxycholate salt, and
saline,
wherein:
the mole ratio of amphotericin B to pegylated-phospholipid is about 1:25 to about 1:200, and
the concentration of pegylated-phospholipid is above its critical micelle concentration.

2. The composition of claim 1 wherein the pegylated-phospholipid is PEG-DSPE.

3. The composition of claim 1 wherein the PEG block of the pegylated-phospholipid has an average molecular weight of about 1,000 Da to about 10,000 Da.

4. The composition of claim 1 wherein the PEG block of the pegylated-phospholipid has an average molecular weight of about 3,000 Da to about 7,000 Da.

5. The composition of claim 1 wherein the PEG block of the pegylated-phospholipid has an average molecular weight of about 5,000 Da.

6. The composition of claim 1 wherein the composition comprises about 0.01 mg/mL to about 1 mg/mL amphotericin B.

7. The composition of claim 1 wherein the composition comprises about 0.05 mg/mL to about 0.2 mg/mL amphotericin B.

8. The composition of claim 1 comprising about 0.01 mg/mL to about 1 mg/mL of the water soluble deoxycholate salt.

9. The composition of claim 8 comprising about 0.04 mg/mL to about 0.2 mg/mL of the water soluble deoxycholate salt.

10. The composition of claim 1 wherein the water soluble deoxycholate salt is deoxycholate sodium salt.

11. The composition of claim 1 wherein the mole ratio of amphotericin B to pegylated phospholipid is about 1:30 to about 1:100.

12. The composition of claim 1 wherein the saline comprises about 0.5 to about 1.5 wt % sodium chloride.

13. The composition of claim 1 further comprising buffer salts.

14. The composition of claim 13 wherein the buffer salts comprise phosphate salts.

15. A composition comprising:
a pegylated-phospholipid selected from PEG-DSPE, wherein the PEG block has an average molecular weight of about 3000 Da to about 7000 Da;
about 0.05-0.2 mg/mL amphotericin B;
about 0.05-0.2 mg/mL deoxycholate sodium salt; and
saline having a concentration of 0.5 wt % to 1.5 wt % sodium chloride;
wherein
the mole ratio of amphotericin B to PEG-DSPE is about 1:30 to 1:100, and
the concentration of PEG-DSPE is above its critical micelle concentration.

16. The composition of claim 15 wherein the saline has a concentration of about 0.9 wt % sodium chloride.

17. The composition of claim 1 further comprising 5-fluorocytosine.

18. The composition of claim 17, wherein the 5-fluorocytosine has a concentration of about 1 mg/mL to about 20 mg/mL.

19. A method of preparing a composition of claim 1 comprising combining a saline solution of the pegylated-phospholipid with an aqueous mixture of the amphotericin B and the water soluble salt of the deoxycholate to provide the concentration of pegylated-phospholipid above its critical micelle concentration and the stated mole ratio of amphotericin B to pegylated-phospholipid.

20. The method of claim 19 further comprising combining an aqueous solution of 5-fluorocytosine with any of the saline solution of the pegylated-phospholipid, the aqueous mixture of the amphotericin B and the water soluble salt of the deoxycholate, or the combined pegylated-phospholipid and amphotericin B mixture.

21. A method of treatment comprising administering an effective amount of the composition of claim 1 to a mammal suffering from a fungal infection.

22. The method of claim 21 wherein the mammal is a human.

23. The method of claim 21 wherein the mammal is infected by one or more of Candida species, Histoplasma capsulatum, Coccidioides immitis, Blastomyces dermatitidis, Rhodotorula, Cryptococcus neoformans, Sporothrix schenckii, Mucor mucedo, and Aspergillus fumigatus.

24. The method of claim 23 wherein the mammal is infected by Candida albicans.

25. A kit for reformulating amphotericin B comprising a first package, a second package and instructions for reformulation, wherein
the first package comprises at least solid ampohotericin B in admixture with a solid water soluble deoxycholate salt;
the second package comprises at least a saline solution of a pegylated phospholipid;
the pegylated phospholipid is at a concentration above its critical micelle concentration; and
the mole ratio of amphotericin B to pegylated-phospholipid upon combining them to form a composition of any one of claims 1-10 or 12-17 is about 1:25 to 1:200, or to form a composition of claim 11 is about 1:30 to about 1:100.

26. The kit of claim 25 further comprising a third package, wherein the third package comprises 5-fluorocystosine.
